(19) United States
(12) Patent Application Publication
(10) Pub. No.: US 2025/0135387 A1
(43) Pub. Date: May 1, 2025

(54) VENTILATION APPARATUS

(71) Applicant: Samsung Electronics Co., Ltd., Suwon-si (KR)

(72) Inventors: Jongwook JOO, Suwon-si (KR); Youngchul KO, Suwon-si (KR); Kyunghoon KIM, Suwon-si (KR); Donghyo KIM, Suwon-si (KR); Myungju SHIN, Suwon-si (KR); Eomji JANG, Suwon-si (KR); Jaehyoung SIM, Suwon-si (KR)

(21) Appl. No.: 19/003,276

(22) Filed: Dec. 27, 2024

Related U.S. Application Data
(63) Continuation of application No. PCT/KR2023/010286, filed on Jul. 18, 2023.

(30) Foreign Application Priority Data
Aug. 23, 2022 (KR) 10-2022-0105828

(51) Int. Cl. B01D 46/00; A61L 2/10; B01D 46/42; B01D 46/52; F24F 8/108; F24F 12/00

(52) U.S. Cl. CPC B01D 46/0028; A61L 2/10; B01D 46/0047; B01D 46/4263; B01D 46/521; F24F 8/108; F24F 12/006; A61L 2202/11; B01D 2273/30; B01D 2279/50; B01D 2279/65

(57) ABSTRACT

A ventilation apparatus is provided. The ventilation apparatus includes a housing, an inlet formed on one side of the housing so outside air is introduced into the housing, an outlet formed on another side of the housing so the air introduced into the housing through the inlet is discharged indoors, a total heat exchanger arranged between the inlet and the outlet, and including an intake inlet end into which air introduced through the inlet and flowing toward the outlet is introduced, a filter arranged inside the housing and arranged between the inlet and the total heat exchanger, and a sterilizing lamp configured to sterilize the filter, wherein the filter includes a first side extending in a vertical direction and a second side extending in a direction perpendicular to the first side, and wherein the sterilizing lamp is arranged between one end of the filter and the intake inlet end.

### CROSS-REFERENCE TO RELATED APPLICATION(S)

This application is a continuation application, claiming priority under 35 U.S.C. § 365 (c), of an International application No. PCT/KR2023/010286, filed on Jul. 18, 2023, which is based on and claims the benefit of a Korean patent application number 10-2022-0105828, filed on Aug. 23, 2022, in the Korean Intellectual Property Office, in the Korean Intellectual Property Office, the disclosure of which is incorporated by reference herein in its entirety.

### BACKGROUND

### 1. Field

The disclosure relates to a ventilation apparatus. More particularly, the disclosure relates to a ventilation apparatus including a sterilizing lamp capable of sterilizing a filter.

### 2. Description of Related Art

A ventilation apparatus is an apparatus that ventilates an indoor space by supplying outdoor air to indoors or exchanging indoor air with outdoor air.

In general, a ventilation apparatus may include an outdoor intake port and an indoor exhaust port that are provided such that outdoor air flows indoors, and an indoor intake port and an outdoor exhaust port that are provided such that indoor air flows outdoors.

A ventilation apparatus may include a total heat exchanger, a high-efficiency particulate air (HEPA) filter, and a pre-filter that are provided for heat exchange between outdoor air and indoor air.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide a ventilation apparatus including a sterilizing lamp, capable of minimizing flow resistance in a path through which outdoor air flows.

Another aspect of the disclosure is to provide a ventilation apparatus including a sterilizing lamp and a reflector arranged to ensure uniform sterilization toward a filter.

Additional aspects will be set forth in part in the description which follows and, in part, will be apparent from the description, or may be learned by practice of the presented embodiments.

In accordance with an aspect of the disclosure, a ventilation apparatus is provided. The ventilation apparatus includes a housing, an inlet formed on one side of the housing such that outside air is introduced into the housing, an outlet formed on another side of the housing such that the air introduced into the housing through the inlet is discharged to indoors, a total heat exchanger arranged between the inlet and the outlet, and including an intake inlet end into which air introduced through the inlet and flowing toward the outlet is introduced, a filter arranged inside the housing and arranged between the inlet and the total heat exchanger, and a sterilizing lamp configured to sterilize the filter, wherein the filter includes a first side extending in a vertical direction and a second side extending in a direction perpendicular to the first side, and wherein the sterilizing lamp is arranged between one end of the filter in a direction in which the second side extends and one end of the intake inlet end in the direction in which the second side extends.

In accordance with another aspect of the disclosure, the sterilizing lamp extends in a direction in which the first side of the filter extends.

In accordance with another aspect of the disclosure, the filter includes a plurality of crests extending toward the total heat exchanger and a plurality of troughs arranged between the plurality of crests, and the plurality of crests and the plurality of troughs extend in the direction in which the second side extends.

In accordance with another aspect of the disclosure, the ventilation apparatus further includes a reflector configured to reflect light emitted from the sterilizing lamp to the filter, being bent to surround the sterilizing lamp and extending along the direction in which the first side extends.

In accordance with another aspect of the disclosure, the reflector includes a first reflector, a second reflector bent from one side of the first reflector, and a third reflector bent from another side of the first reflector.

In accordance with another aspect of the disclosure, the first reflector and the third reflector are arranged to form an obtuse angle with each other.

In accordance with another aspect of the disclosure, an angle formed by the first reflector and the second reflector is between 70 degrees and 110 degrees, and an angle formed by the first reflector and the third reflector is between 110 degrees and 165 degrees.

In accordance with another aspect of the disclosure, the sterilizing lamp includes a cylindrical shape, and when a vertical distance between a center of the sterilizing lamp and the filter is denoted as h, and a length of the first reflector in a direction perpendicular to the extension direction of the first side is denoted as n and a length of the second reflector is denoted as p, n/h and p/h may each be provided between 0.05 and 0.2.

In accordance with another aspect of the disclosure, the sterilizing lamp includes a cylindrical shape, and when a vertical distance between a center of the sterilizing lamp and the filter is denoted as h, and a length of the third reflector in a direction perpendicular to the extension direction of the first side is denoted as q, q/h may be provided between 0.08 and 0.4.

In accordance with another aspect of the disclosure, the sterilizing lamp includes a cylindrical shape, and when a vertical distance between a center of the sterilizing lamp and the filter is denoted as h, and a length of the second side of the filter is denoted L, h/L may be provided between 0.15 and 0.6.

In accordance with another aspect of the disclosure, the sterilizing lamp includes a cylindrical shape, and when a vertical distance between a center of the sterilizing lamp and the filter is denoted as h, a vertical distance between a vertical line from the center of the sterilizing lamp to an extension line of the second side of the filter and an end of the filter adjacent to the vertical line is denoted as m, h/m may be provided between −0.2 and 0.5.

In accordance with another aspect of the disclosure, the ventilation apparatus further includes a front frame including an opening that may be open toward the filter, and a rear frame configured to support the front frame and the reflector and couplable to the housing.

In accordance with another aspect of the disclosure, the rear frame includes a reflective support plate formed to correspond to a bent shape of the reflector to support the reflector.

In accordance with another aspect of the disclosure, the ventilation apparatus further includes a grid frame provided in the opening.

In accordance with another aspect of the disclosure, the housing includes an inner wall arranged at one side of the housing adjacent to the inlet and including a fixing groove into which one side of the rear frame adjacent to the filter may be inserted.

In accordance with another aspect of the disclosure, a ventilation apparatus is provided. The ventilation apparatus includes a housing allowing outside air to flow to indoors and allowing inside air to flow outside, an inlet formed on one side of the housing such that the outside air is introduced into the housing, an outlet formed on another side of the housing such that the air introduced into the housing through the inlet is discharged to indoors, a total heat exchanger arranged between the inlet and the outlet, a filter between the inlet and the total heat exchanger, and a sterilizing lamp configured to sterilize the filter, wherein the filter includes a first side extending in a vertical direction and a second side extending in a direction perpendicular to the first side, and the sterilizing lamp extends in a direction in which the first side extends.

The housing includes an inner wall arranged at one side of the housing adjacent to the inlet, and the sterilizing lamp may be arranged adjacent to the inner wall.

The filter includes a plurality of crests extending toward the total heat exchanger and a plurality of troughs arranged between the plurality of crests, and the plurality of crests and the plurality of troughs may extend in a direction in which the second side extends.

The ventilation apparatus further includes a reflector arranged between the sterilizing lamp and the inner wall of the housing to reflect light emitted from the sterilizing lamp to the filter, the reflector being bent to surround the sterilizing lamp and extending along the direction in which the first side extends.

In accordance with another aspect of the disclosure, a ventilation apparatus is provided. The ventilation apparatus includes a housing, an inlet formed on one side of the housing such that outside air is introduced into the housing, an outlet formed on another side of the housing such that the air introduced into the housing through the inlet is discharged to indoors, a filter configured to pass the air introduced into the housing through the inlet, and including a first side extending in a vertical direction and a second side extending in a direction perpendicular to the first side, a total heat exchanger arranged between the filter and the outlet in a flow direction of the air introduced into the housing through the filter and including an intake inlet end into which the air is introduced, a sterilizing lamp extending in a direction in which the first side of the filter extends, and a reflector bent to surround the sterilizing lamp and extending along the direction in which the first side extends.

According to an aspect of the disclosure, the ventilation apparatus includes a sterilizing lamp disposed between a filter and a total heat exchanger, allowing the filter in the ventilation apparatus to be sterilized.

According to another aspect of the disclosure, the sterilizing lamp does not interfere with the airflow between the filter and the total heat exchanger, enabling smooth airflow.

According to another aspect of the disclosure, the extension directions of the sterilizing lamp and the filter intersect with each other, thereby providing the ventilation apparatus with relatively uniform irradiation in the troughs between the crests of the filter.

According to another aspect of the disclosure, the arrangement of the sterilizing lamp and the reflector can ensure uniform irradiance intensity of light emitted from the sterilizing lamp to the filter.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a perspective view illustrating a ventilation apparatus according to an embodiment of the disclosure;

FIG. 2 is an exploded perspective view of the ventilation apparatus with a first cover separated according to an embodiment of the disclosure;

FIG. 3 is a perspective view illustrating the ventilation apparatus shown in FIG. 2, when viewed at a different angle according to an embodiment of the disclosure;

FIG. 4 is a cross-sectional view of the ventilation apparatus shown in FIG. 1 according to an embodiment of the disclosure;

FIG. 5 is an enlarged view of portion A shown in FIG. 4 according to an embodiment of the disclosure;

FIGS. 6, 7, and 8 are schematic views of a sterilizing lamp and a filter according to various embodiments of the disclosure;

FIG. 9 is a schematic view of a sterilizing lamp and a reflector according to an embodiment of the disclosure;

FIG. 10 is a perspective view illustrating an ultraviolet (UV)-C module according to an embodiment of the disclosure;

FIG. 11 is an exploded perspective view of the UVC module FIG. 10 according to an embodiment of the disclosure;

FIG. 12 is a cross-sectional view taken along line I-I′ of FIG. 10 according to an embodiment of the disclosure;

FIG. 13 is a cross-sectional view taken along line II-II′ of FIG. 10 according to an embodiment of the disclosure;

FIG. 14 is a drawing illustrating a fiber material of a filter inoculated with a bacterial solution to show a sterilization experiment with a structure according to an embodiment of the disclosure; and

FIG. 15 is a drawing comparing the amount of light when the fiber material of the filter shown in FIG. 14 is irradiated with a sterilizing lamp and the amount of light when irradiated with a sterilizing lamp and a reflector according to an embodiment of the disclosure.

The same reference numerals are used to represent the same elements throughout the drawings.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

In the description of the drawings, like numbers refer to like elements throughout the description of the drawings.

The terms used herein are for the purpose of describing the embodiments and are not intended to restrict and/or to limit the disclosure. In addition, the terms “comprises”, “includes”, and “has” are intended to indicate that there are features, numbers, steps, operations, elements, parts, or combinations thereof described in the disclosure, and do not exclude the presence or addition of one or more other features, numbers, steps, operations, elements, parts, or combinations thereof.

It will be understood that, although the terms first, second, etc. used in the disclosure may be used herein to describe various components, these components should not be limited by these terms. These terms are only used to distinguish one component from another. For example, a first element could be termed a second element, and similarly, a second element could be termed a first element without departing from the scope of the disclosure. The term “and/or” includes combinations of one or all of a plurality of associated listed items.

Further, as used in the disclosure, the terms “front”, “rear”, “top”, “bottom”, “side”, “left”, “right”, “upper”, “lower”, and the like are defined with reference to the drawings, and are not intended to limit the shape and position of each component.

It should be appreciated that the blocks in each flowchart and combinations of the flowcharts may be performed by one or more computer programs which include instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g. a central processing unit (CPU)), a communication processor (CP, e.g., a modem), a graphics processing unit (GPU), a neural processing unit (NPU) (e.g., an artificial intelligence (AI) chip), a Wi-Fi chip, a Bluetooth® chip, a global positioning system (GPS) chip, a near field communication (NFC) chip, connectivity chips, a sensor controller, a touch controller, a finger-print sensor controller, a display driver integrated circuit (IC), an audio CODEC chip, a universal serial bus (USB) controller, a camera controller, an image processing IC, a microprocessor unit (MPU), a system on chip (SoC), an IC, or the like.

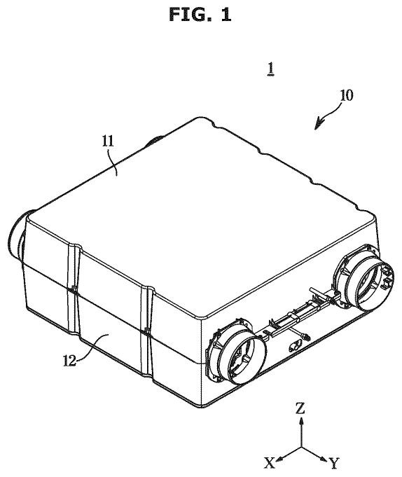

FIG. 1 is a perspective view illustrating a ventilation apparatus 1 according to an embodiment of the disclosure.

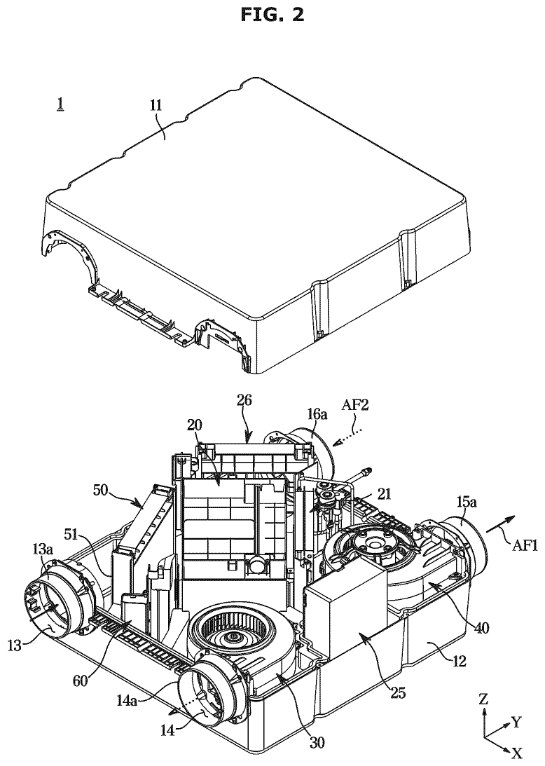

FIG. 2 is an exploded perspective view of the ventilation apparatus with a housing 10 separated according to an embodiment of the disclosure.

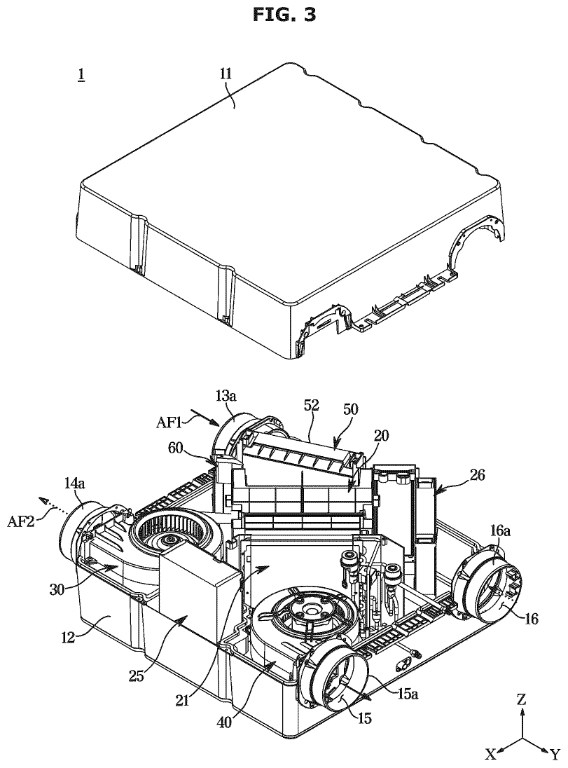

FIG. 3 is a perspective view illustrating the ventilation apparatus 1 shown in FIG. 2, when viewed at a different angle according to an embodiment of the disclosure.

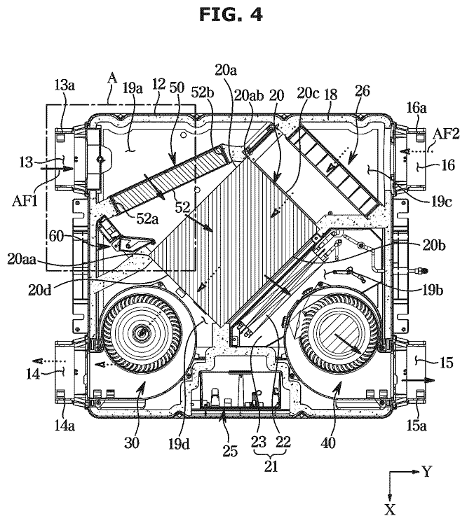

FIG. 4 is a cross-sectional view of the ventilation apparatus shown in FIG. 1 according to an embodiment of the disclosure.

Referring to FIGS. 1 to 4, the ventilation apparatus 1 may include a housing 10 forming an external appearance. The housing 10 may be provided in a roughly box shape. The housing 10 may include an intake airflow path AF1 for drawing outdoor air into the indoors, and an exhaust airflow path AF2 for discharging indoor air to the outdoors.

The ventilation apparatus 1 may be arranged inside an indoor space to introduce outdoor air into the indoor space and discharge indoor air to the outdoor space, thereby ventilating the indoor space. Also, it may circulate indoor air.

When the ventilation apparatus is arranged in an indoor space such as a multi-family house or an apartment, the indoor space may be provided as a pair of indoor spaces.

The ventilation apparatus 1 may include covers 11 and 12. The covers 11 and 12 may include a first cover 11 arranged at the top in the vertical direction Z, and a second cover 12 arranged at the bottom of the first cover 11 in the vertical direction Z and coupled to the first cover 11 in the vertical direction Z.

The housing 10 may include an outdoor intake port 13 that is connected to an outdoor space and through which outdoor air is drawn into the housing 10, and an indoor exhaust port 15 that is connected to an indoor space and through which air inside the housing 10 is exhausted to the indoors.

The housing 10 may include an indoor intake port 16 that is connected to an indoor space and through which indoor air is drawn into the housing 10, and an outdoor exhaust port 14 that is connected to an outdoor space and through which air inside the housing 10 is exhausted to the outdoors.

The outdoor air flowing into the housing 10 through the outdoor intake port 13 may flow along the intake airflow path AF1, which flows to the indoors through the indoor exhaust port 15.

The indoor air flowing into the housing 10 through the indoor intake port 16 may flow along the exhaust airflow path AF2, which flows to the outdoors through the outdoor exhaust port 14.

The outdoor intake port 13 may be an inlet 13 formed on one side of the housing 10 such that outdoor air may flow into the housing 10. The indoor exhaust port 15 may be an outlet 15 formed on the other side of the housing 10 such that air flowing into the housing 10 through the inlet 13 may be discharged toward the indoors.

The housing 10 may include an outdoor intake duct 13a arranged adjacent to the outdoor intake port 13 to guide outdoor air to the inside of the housing 10. The housing 10 may include an indoor exhaust duct 15a arranged adjacent to the indoor exhaust port 15 to guide outdoor air introduced into the housing 10 to the indoor space.

The housing 10 may include an indoor intake duct 16a arranged adjacent to the indoor intake port 16 to guide indoor air into the housing 10. The housing 10 may include an outdoor exhaust duct 14a arranged to guide indoor air introduced into the housing 10 to the outdoor space.

The ventilation apparatus 1 may include a first blower 40 arranged inside the housing 10 and generating a blowing force required to draw outdoor air into the indoor space while communicating with the indoor exhaust port 15. The ventilation apparatus 1 may include a second blower 30 arranged inside the housing 10 and generating a blowing force required to discharge indoor air to the outdoors while communicating with the outdoor exhaust port 14.

The ventilation apparatus 1 may include a total heat exchanger 20 that is arranged to allow heat exchange between air flowing through the exhaust airflow path AF2 and air flowing through the intake airflow path AF1.

The total heat exchanger 20 may correspond to a plate-type total heat exchanger or a rotary-type total heat exchanger. The total heat exchanger 20 may be arranged at a point in which the intake airflow path AF1 and the exhaust airflow path AF2 intersect with each other. In other words, the total heat exchanger 20 may be arranged on the intake airflow path AF1 and the exhaust airflow path AF2 simultaneously.

The total heat exchanger 20 may connect a first intake chamber 19a and a second intake chamber 19b. The total heat exchanger 20 may connect a first exhaust chamber 19c and a second exhaust chamber 19d.

The total heat exchanger 20 may include an intake inlet end 20a adjacent to the first intake chamber 19a and into which air flowing within the first intake chamber 19a is introduced. The total heat exchanger 20 may include an intake discharge end 20b adjacent to the second intake chamber 19b and which discharges air introduced through the intake inlet end 20a into the second intake chamber 19b.

The total heat exchanger 20 may include an exhaust inlet end 20c adjacent to the first exhaust chamber 19c and into which air flowing within the first exhaust chamber 19c is introduced. The total heat exchanger 20 may include an exhaust discharge end 20d adjacent to the second exhaust chamber 19d and which discharges air introduced through the exhaust inlet end 20c into the second exhaust chamber 19d.

The ventilation apparatus 1 may include a filter 50 provided to remove foreign substances flowing in the outdoor air.

The filter 50 may be provided to collect foreign substances of a certain size. The filter 50 may be a high efficiency particulate air (HEPA) filter that collects fine dust of a certain size. The HEPA filter may be composed of glass fiber.

However, the filter 50 is not limited thereto, and the filter 50 may be provided as various types of filters that collect foreign substances.

In addition, the filter 50 is not limited thereto, and may be provided as a photocatalytic filter that induces a chemical reaction of air using a photocatalyst. That is, the filter 50 includes a photocatalyst, and may sterilize various pathogens and bacteria present in the air by inducing a chemical reaction with the light energy of the photocatalyst. By promoting the chemical reaction, odor particles in the air may be decomposed, removed, or captured.

The filter 50 may be arranged between the total heat exchanger 20 and the inlet 13. The filter 50 may include a first side 51 extending in the vertical direction Z and a second side 52 extending in a direction perpendicular to the first side 51. The filter 50 may have an approximately rectangular parallelepiped shape.

The filter 50 may be arranged to face the intake inlet end 20a of the total heat exchanger 20. The filter 50 may be arranged adjacent to the inlet 13 inside the housing 10 and may be arranged between the inlet 13 and the total heat exchanger 20.

Preferably, the filter 50 may be arranged to closely face the intake inlet end 20a. Accordingly, foreign substances flowing in outdoor air introduced through the outdoor intake port 13 may be collected by the filter 50 before the outdoor air flows into the total heat exchanger 20, thereby preventing the total heat exchanger 20 from being contaminated.

More specifically, the filter 50 may be arranged to have one side adjacent to the inlet 13 in parallel with the other side adjacent to the total heat exchanger 20.

For this structure, the filter 50 may be extended such that outdoor air flowing into the housing 10 through the inlet 13 passes the filter 50. More specifically, the filter 50 may be extended to partition the first intake chamber 19a such that outdoor air flowing into the housing 10 through the inlet 13 passes through the filter 50. The filter 50 may be extended from one side of the housing 10 toward the other side of the housing 10.

The ventilation apparatus 1 may include a dehumidifying unit 21 configured to control the humidity of the air flowing through the intake airflow path.

The dehumidifying unit 21 may include a heat exchanger 22 configured to exchange heat with outdoor air flowing inside the intake airflow path, and a drain tray 23 configured to collect condensate generated in the heat exchanger 22.

The heat exchanger 22 may be configured as a pair of heat exchangers. Each of the pair of heat exchangers 22 may exchange heat with outdoor air. The pair of heat exchangers 22 may operate in a mode in which both heat exchangers serve as evaporators, or in a mode in which one heat exchanger serves as an evaporator and the other heat exchanger serves as a condensate.

As described above, the heat exchanger 22 may operate in various modes to selectively dehumidify the outdoor air while maintaining the temperature of the outdoor air or to dehumidify the outdoor air while cooling the outdoor air.

However, the disclosure is not limited thereto, and the heat exchanger 22 may be provided as a single heat exchanger to mainly dehumidify the outdoor air.

The dehumidifying unit 21 may be provided on the intake airflow path AF1. The dehumidifying unit 21 may be arranged inside the second intake chamber 19b. That is, the dehumidifying unit 21 may be arranged downstream of the total heat exchanger 20 in the intake airflow path AF1.

The ventilation apparatus 1 may include a control box 25 arranged inside the housing 10. The control box 25 may include a power supply configured to supply power to printed circuit board (PCB) elements and the ventilation apparatus 1.

The ventilation apparatus 1 may include a pre-filter 26 arranged adjacent to the indoor intake port 16. The pre-filter 26 may be provided to remove foreign substances flowing in the indoor air. The pre-filter 26 may be arranged between the exhaust inlet end 20c of the total heat exchanger 20 and the indoor intake port 16.

The ventilation apparatus 1 may include an ultraviolet (UV)-C module 60 provided to sterilize the filter 50. The UVC module 60 may be a sterilization module 60. The UVC module 60 may be a configuration to irradiate the filter 50 with ultraviolet wavelengths in the range of 200 nm to 280 nm. More specifically, the UVC module 60 may be configured to sterilize bacteria or viruses that may breed in the filter 50 by 99.9% using environmentally friendly light that does not contain chemicals or mercury. The UVC from the UVC module 60 may be referred to as light hereinafter. The light may damage the bacterial DNA and RNA molecular structures, thereby preventing the bacteria from reproducing.

The UVC module 60 may be arranged between the filter 50 and the total heat exchanger 20. More specifically, the UVC module 60 may be arranged to face the filter 50.

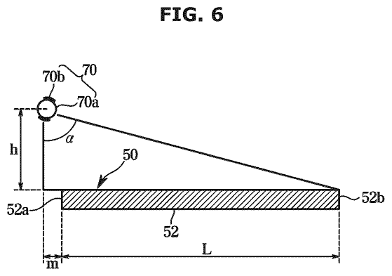

The UVC module 60 may be extended in the vertical direction Z. The UVC module 60 may include a sterilizing lamp 70, see FIG. 6 that may be extended in the vertical direction Z.

More specifically, the UVC module 60 may be arranged to face the other side of the filter 50 adjacent to the total heat exchanger 20. In this case, when the UVC module 60 is arranged on a path through which outdoor air flows, it may interfere with the flow of air. To prevent this, the UVC module 60 may be arranged adjacent to one side of the housing 10. Here, the one side of the housing 10 may be one side adjacent to the inlet 13.

The filter 50 may include one end 52a and the other end 52b in the extension direction of the second side 52. One end 52a and the other end 52b of the second side 52 may be the one end 52a and the other end 52b of the filter 50. The intake inlet end 20a of the total heat exchanger 20 may include one end 20aa and the other end 20ab in the extension direction of the second side 52 of the filter 50.

Here, the one end 52a of the filter 50 may be arranged closer to the one end 20aa of the intake inlet end 20a than to the other end 20ab of the intake inlet end 20a. In addition, the other end 52b of the filter 50 may be arranged closer to the other end 20ab of the intake inlet end 20a than to the one end 20aa of the intake inlet end 20a.

The UVC module 60 may be arranged between the one end 52a of the filter 50 and the one end 20aa of the intake inlet end 20a. However, the arrangement of the UVC module 60 is not limited thereto, and may be arranged between the other end 52b of the filter 50 and the other end 20ab of the intake inlet end 20a.

According to such a structure, the arrangement of the UVC module 60 may prevent the air flowing from the filter 50 to the total heat exchanger 20 from being disturbed while the filter 50 may be sterilized.

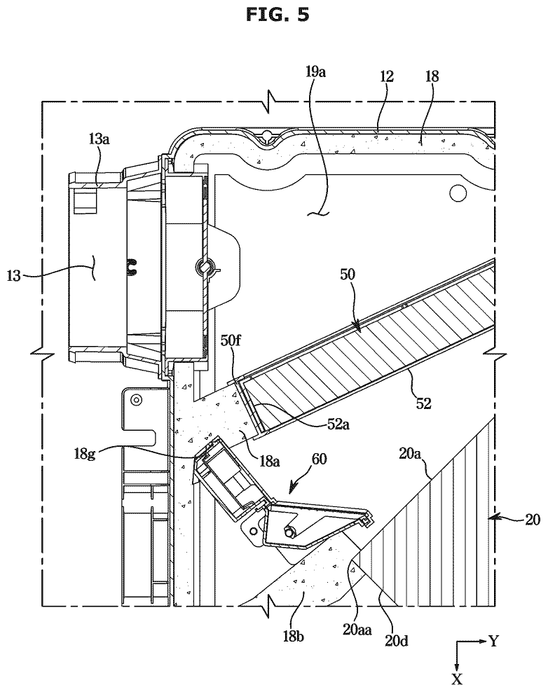

FIG. 5 is an enlarged view of portion A shown in FIG. 4 according to an embodiment of the disclosure.

Referring to FIG. 5, an inner wall 18 may include a first inner wall 18a and a second inner wall 18b protruding toward the first intake chamber 19a.

The housing 10 may include the inner wall 18 formed of an insulating material. Preferably, the inner wall 18 may be formed of an expanded polystyrene (EPS) insulating material, such as Styrofoam. However, the disclosure is not limited thereto, and the inner wall 18 may be formed of various insulating materials such that the temperature of the air flowing through the intake airflow path AF1 and the exhaust airflow path AF2 may be maintained at a constant temperature.

The covers 11 and 12, see FIG. 1 may be provided to cover the inner wall 18 formed of an insulating material to protect the inner wall 18 from the outside. The covers 11 and 12 may be formed of an injection molded product, such as plastic.

The first inner wall 18a may protrude toward the first intake chamber 19a at a portion adjacent to the inlet 13 to fix the filter 50, and the second inner wall 18b may protrude toward the total heat exchanger 20 at a portion adjacent to the inlet 13.

A fixing member 50f may be provided between the first inner wall 18a and the filter 50. The first inner wall 18a may include a fixing groove 18g into which one side of the UVC module 60 adjacent to the inlet 13 is inserted. According to such a structure, the UVC module 60 may be stably supported by the fixing groove 18g, thereby preventing the flow of air passing through the filter 50 from interfering while the UVC module 60 may be arranged between the total heat exchanger 20 and the filter 50.

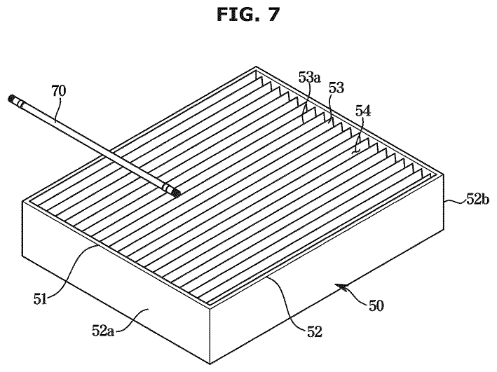

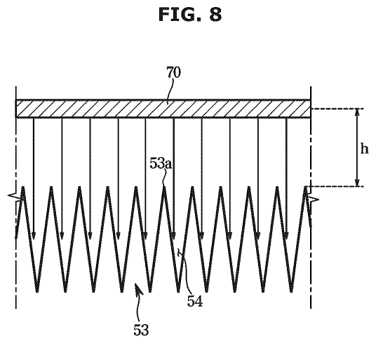

FIGS. 6 to 8 are schematic diagrams of a sterilizing lamp 70 and a filter 50 according to various embodiments of the disclosure.

Referring to FIGS. 6 to 8, the sterilizing lamp 70 may include a cylindrical shape.

The sterilizing lamp 70 may be extended in the extension direction of the first side 51 of the filter 50. More specifically, the sterilizing lamp 70 may be extended in the vertical direction Z with reference to FIG. 1, and this direction may be a direction perpendicular to the extension direction of the second side 52 of the filter 50.

The sterilizing lamp 70 may include a lamp body 70a having a cylindrical shape and an external electrode 70b that may be attached to the outer surface of the lamp body 70a. The lamp body 70a may include a gas (not shown) that emits ultraviolet rays through discharge.

The material of the lamp body 70a may be quartz or borosilicate glass for efficient transmission of ultraviolet rays. The lamp body 70a may be provided in a cylindrical shape and may extend in the vertical direction Z. The lamp body 70a may be provided in a tube shape with both ends closed. A plurality of cap electrodes (not shown) may be provided and fixed to each end of the lamp body 70a.

The lamp body 70a may include a discharge space that is hollowed inside. A gas that emits ultraviolet rays through discharge may be accommodated inside the lamp body 70a. A noble gas or the like may be used as a gas that emits ultraviolet rays through a discharge. When voltage is applied to a gas such as a noble gas in the ground state, an excited dimer molecule (hereinafter referred to as ‘excimer (excited dimer)’) is generated. Since the excimer is in an unstable state, it immediately returns to the ground state, emitting ultraviolet rays. The gas to be filled inside the lamp body 70a may be provided using xenon (Xe or a xenon mixture gas (Xe, Ar, Ne, etc.). The inner wall of the lamp body 70a may be coated with a phosphor (not shown) that may convert the emission wavelength of xenon 172 nm) into a sterilizing wavelength range of 250 nm to 260 nm.

However, the gas accommodated inside the lamp body 70a is not limited to a rare gas, such as xenon and a mixed gas of rare gases, and the gas may be provided in various types as long as it can emit light in the ultraviolet range through discharge. The lamp body 70a may accommodate a fluorescent substance inside, depending on the wavelength of the ultraviolet light emitted.

In order to implement an external electrode fluorescent light (EEFL) type lamp, an external electrode 70b need to be arranged on the surface of the lamp body 70a. The external electrode 70b may be arranged on the outer surface of the lamp body 70a and extend along the vertical direction (e.g., the entering direction in FIG. 6 in which the lamp body 70a extends. That is, the external electrode 70b may be arranged on the outer surface of the lamp body 70a and may extend in the longitudinal direction of the lamp body 70a.

The external electrode 70b may include a plurality of external electrodes 70b. Specifically, the plurality of external electrodes 70b may be provided as a pair of external electrodes 70b, and may be arranged on the outer surface of the lamp body 70a to face each other with respect to the central axis of the lamp body 70a.

The external electrode 70b may be provided in the form of a tape on the surface of the lamp body 70a. The material of the external electrode 70b is not particularly limited as long as it has conductivity, and may be provided as, for example, gold, silver, nickel, carbon, gold palladium, silver palladium, platinum, aluminum, etc., or an alloy thereof.

The center of the sterilizing lamp 70 may be refer to the central axis of the lamp body 70a. The vertical distance between the center of the sterilizing lamp 70 and the filter 50 may be h. Here, the vertical distance h may be the vertical distance between the sterilizing lamp 70 and one side of the filter 50 adjacent to the sterilizing lamp 70. In addition, the vertical distance h may be a vertical distance from the center of the sterilizing lamp 70 to a point on an extension line of the filter 50 along the second side 52 that is the closest to the sterilizing lamp 70.

The length of the extension direction of the second side 52 of the filter 50 may be L. In this case, the vertical distance h between the center of the sterilizing lamp 70 and the filter 50 with respect to the length (L) of the filter 50 may be in a range of 0.15 and 0.6. That is, h/L may be set between 0.15 and 0.6.

A vertical distance between a vertical line from the center of the sterilizing lamp 70 to an extension line of the filter 50 in the extension direction of the second side 52 and an end 52a of the sterilizing lamp 70 adjacent to the vertical line may be m.

In this case, the vertical distance h between the center of the sterilizing lamp 70 and the filter 50 with respect to the vertical distance m between the vertical line from the center of the sterilizing lamp 70 to the extension line of the filter 50 and the end 52a of the filter 50 adjacent to the vertical line may be in a range of −0.2 and 0.5.

That is, h/m may be set between −0.2 and 0.5. According to such a structure, the angle α between the vertical line of the sterilizing lamp 70 and an auxiliary line connecting the sterilizing lamp 70 to the other end 52b of the filter 50 may be approximately 72°.

The filter 50 may include a plurality of crests 53 that protrude to face the sterilizing lamp 70, extend along the extension direction of the filter 50, and are arranged in a direction intersecting with the extension direction of the filter 50.

The plurality of crests 53 may have a shape that protrudes toward one side of the filter 50 adjacent to the inlet 13, see FIG. 4 and the other side adjacent to the total heat exchanger 20. The plurality of crests 53 may include a ‘V’ shape. Ends 53a of the plurality of crests 53 may be the apices pointing toward the inlet 13.

The vertical distance h from the center of the sterilizing lamp 70 to the filter 50 described above may also be defined as the shortest vertical distance from the center of the sterilizing lamp 70 to the ends 53a of the plurality of crests 53 on a vertical line between the sterilizing lamp 70 and the filter 50.

A plurality of troughs 54 may be formed between the plurality of crests 53. The plurality of crests 53 may be convex portions, and the plurality of troughs 54 may be concave portions.

The plurality of crests 53 and the plurality of troughs 54 may be extended along the extension direction of the second side 52 of the filter 50 and may be arranged in the extension direction of the first side 51 of the filter 50. The sterilizing lamp 70 may be extended in the direction in which the plurality of crests 53 are arranged. In other words, the sterilizing lamp 70 may be extended in the direction in which the plurality of troughs 54 are arranged.

When the sterilizing lamp 70 is arranged in the direction in which the plurality of crests 53 are extended, the sterilizing power may be high at the plurality of troughs 54 between the plurality of crests 53 adjacent to the sterilizing lamp 70, but the sterilizing power may be reduced at the plurality of troughs 54 between the plurality of crests 53 that are far from the sterilizing lamp 70.

To prevent this, the sterilizing lamp 70 may be extended in the direction in which the plurality of crests 53 are arranged. That is, the structure in which the extension direction of the sterilizing lamp 70 and the extension direction of the plurality of crests 53 are perpendicular to each other may prevent the sterilizing power from being reduced in the plurality of troughs 54 between the plurality of crests 53.

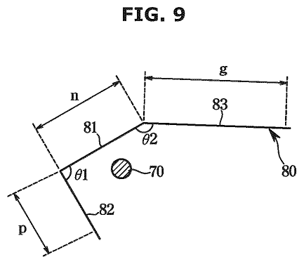

FIG. 9 is a schematic diagram of a sterilizing lamp 70 and a reflector 80 according to an embodiment of the disclosure.

The structure of the sterilizing lamp 70 and the reflector 80 will be described in detail with reference to FIG. 9.

The UVC module 60, see FIG. 5 may include a reflector 80 that is provided to reflect light from the sterilizing lamp 70 to the filter 50. The reflector 80 may be extended along the extension direction of the sterilizing lamp 70.

The reflector 80 may be arranged on a side of the sterilizing lamp 70 that is not facing the filter 50. Such a structure is needed because light of the sterilizing lamp 70 that is directed toward the filter 50 may reach the filter without being reflected but light of the sterilizing lamp 70 that is not directed toward the filter 50 may reach the filter 50 only by being reflected on the reflector 80.

The reflector 80 may be bent. More specifically, the reflector 80 may include a first reflector 81, a second reflector 82 bent from one side of the first reflector 81, and a third reflector 83 bent from the other side of the first reflector 81.

However, the shape of the reflector 80 is not limited thereto, and may be further bent to include a fourth reflector, and the like.

With the arrangement of the filter 50 according to an example, the amount of light emitted from the sterilizing lamp 70 and reaching one end 52a adjacent to the sterilizing lamp 70 of the filter 50 may different from that of the other end 52a distant from the sterilizing lamp 70 of the filter 50. More specifically, a portion of the filter 50 adjacent to the sterilizing lamp 70 may be easily degraded and worn out, and a portion of the filter 50 that is distant from the sterilizing lamp 70 may have insufficient sterilizing power (see FIG. 6.

To prevent such a constraint, the bent reflector 80 may be used to allow the amount of light reaching the portion of the filter 50 adjacent to the sterilizing lamp 70 and the amount of light reaching the portion of the filter 50 distant from the sterilizing lamp 70 to be uniform. In other words, the portion of the filter 50 that is adjacent to the sterilizing lamp 70 may be prevented from being worn out. In addition, the portion of the filter 50 that is distant from the sterilizing lamp 70 may also be prevented from having less sterilizing power.

The first reflector 81 and the second reflector 82 may be perpendicular to each other on a plane that is perpendicular to the direction in which the sterilizing lamp 70 extends. The first reflector 81 and the third reflector 83 may be connected to form an obtuse angle with each other on the plane perpendicular to the extension direction of the sterilizing lamp 70. The second reflector 82 and the third reflector 83 may be arranged to face each other. The first to third reflectors 81 to 83 may be bent to surround the sterilizing lamp 70.

The angle θ1 between the first reflector 81 and the second reflector 82 may be formed between 70 degrees and 110 degrees, and the angle θ2 between the first reflector 81 and the third reflector 83 may be formed between 110 degrees and 165 degrees. The angle θ1 between the first reflector 81 and the second reflector 82 may be set smaller than the angle θ2 between the first reflector 81 and the third reflector 83.

A length of the first reflector 81 in a direction perpendicular to the extension direction of the sterilizing lamp 70 may be n. In this case, the length of the second reflector 82 may be p, and the length of the third reflector 83 may be g.

The length n of the first reflector 81 relative to the vertical distance h between the center of the sterilizing lamp 70 and the filter 50 may be provided in a range of 0.05 to 0.2 (see FIG. 6. That is, n/h may satisfy 0.05<n/h<0.2.

The length p of the second reflector 82 relative to the vertical distance h between the center of the sterilizing lamp 70 and the filter 50 may be provided in a range of 0.05 to 0.2. That is, p/h may be greater than 0.05 and less than 0.2.

The length g of the third reflector 83 relative to the vertical distance h between the center of the sterilizing lamp 70 and the filter 50 may be set in a range of 0.08 to 0.4. That is, g/h may be set at a ratio of 0.08 to 0.4.

According to the structure as described above, light emitted from the sterilizing lamp 70 may uniformly reach the portion of the filter 50 that is adjacent to the sterilizing lamp 70 and the portion of the filter 50 that is distant from the sterilizing lamp 70 due to the shape of the reflector 80.

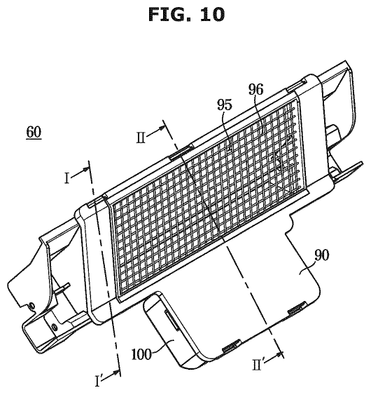

FIG. 10 is a perspective view illustrating an UVC module 60 according to an embodiment of the disclosure.

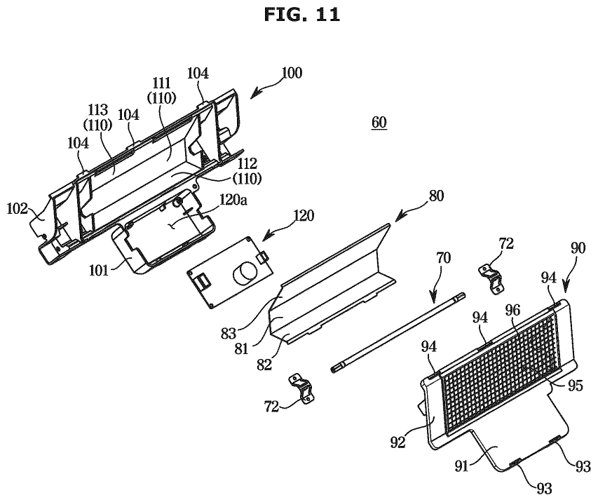

FIG. 11 is an exploded perspective view of the UVC module 60 FIG. 10 according to an embodiment of the disclosure.

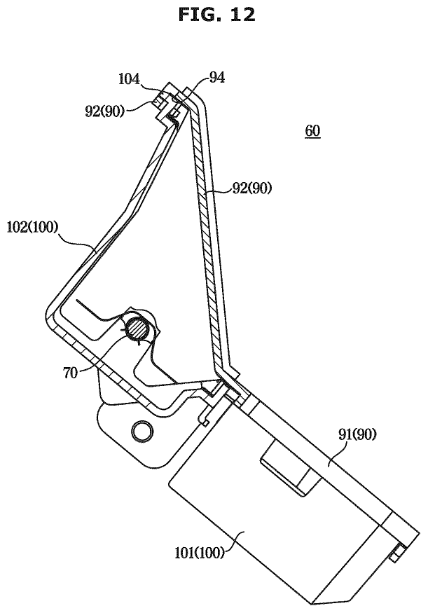

FIG. 12 is a cross-sectional view taken along line I-I′ of FIG. 10 according to an embodiment of the disclosure.

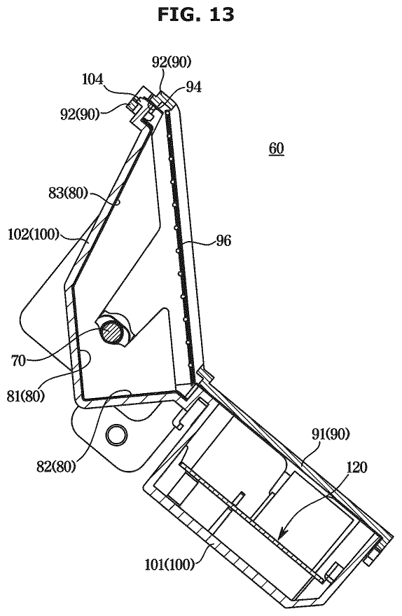

FIG. 13 is a cross-sectional view taken along line II-II′ of FIG. 10 according to an embodiment of the disclosure.

Referring to FIGS. 10 to 13, the UVC module 60 may include a front frame 90 including an opening 95 that is open such that light emitted from the sterilizing lamp 70 may reach the filter 50, see FIG. 4. The front frame 90 may be arranged between the sterilizing lamp 70 and the filter 50. The UVC module 60 may include a grid frame 96 that may prevent a finger from being inserted through the opening 95.

More specifically, when replacing a total heat exchanger 20 disposed within a ventilation apparatus 1, see FIG. 4, there is a need to disengage one of the covers 11 and 12, see FIG. 1 of the ventilation apparatus 1, and replace the total heat exchanger 20. In this case, when a hand or the like that may be adjacent to the UVC module 60 enters toward the sterilizing lamp 70, the human body may be damaged. To prevent this, the grid frame 96 may be provided.

The UVC module 60 may include a rear frame 100 that is configured to support the front frame 90, the sterilizing lamp 70 and the reflector 80.

The UVC module 60 may include an inverter 120 that is provided to apply high voltage to the sterilizing lamp 70. Although not shown in the drawings, the inverter 120 may receive current from the outside and apply high voltage to the external electrode 70b, see FIG. 6 of the sterilizing lamp 70.

The front frame 90 may include a lamp cover 92 having a roughly quadrangular shape and an inverter cover 91 provided below the lamp cover 92. The lamp cover 92 and the inverter cover 91 may be formed as a single body.

The lamp cover 92 may include an opening 95. The lamp cover 92 may be provided at the front of the sterilizing lamp 70. A plurality of coupling grooves 94 may be provided on the upper side of the lamp cover 92. The lamp cover 92 may be coupled to the grid frame 96. The lamp cover 92 may extend in the longitudinal direction of the sterilizing lamp 70.

The inverter cover 91 may have an approximately quadrangular shape and may be provided below the opening 95. A fixing groove 93 may be provided in a lower portion of the inverter cover 91, and a part of the inner wall 18, see FIG. 5 may be inserted into the fixing groove 93.

The rear frame 100 may include an upper rear frame 102 configured to accommodate the sterilizing lamp 70, and a lower rear frame 101 provided at the lower side of the upper rear frame 102.

The upper rear frame 102 may be couplable to the lamp cover 92 and may support the lamp cover 92. The lower rear frame 101 may be couplable to the inverter cover 91 and may support the inverter cover 91.

A plurality of coupling protrusions 104 may be provided on the upper portion of the upper rear frame 102. The coupling protrusions 104 may be inserted into the coupling grooves 94 of the lamp cover 92, and the upper rear frame 102 and the lamp cover 92 may be coupled by the coupling protrusions 104 being inserted into the coupling grooves 94.

Similarly, the lower rear frame 101 may include a locking groove formed adjacent to the inverter cover 91, and the inverter cover 91 may include a locking protrusion protruding toward the lower rear frame 101. The lower rear frame 101 and the inverter cover 91 may be coupled by the locking protrusion being inserted into the locking groove.

According to such a structure, the rear frame 100 and the front frame 90 may be easily coupled to or decoupled from each other. That is, the components such as the sterilizing lamp 70, etc. may be more easily replaced within the UVC module 60.

Lamp fixing members 72 may be arranged on both sides of the sterilizing lamp 70. The lamp fixing members 72 may be configured to fix both ends of the sterilizing lamp 70 to the inside of the upper rear frame 102. The two ends of the sterilizing lamp 70 may be parts that enables coupling with the lamp fixing member 72 without emitting UVC light.

The reflector 80 may be arranged between the sterilizing lamp 70 and the upper rear frame 102. The reflector 80 may be formed to be bent at least once and surround the sterilizing lamp 70 toward the front frame 90. The reflector 80 may include the first reflector 81, the second reflector 82, and the third reflector 83.

The upper rear frame 102 may include a reflective support plate 110 arranged adjacent to the reflector 80. Since the reflector 80 may be bent at least once, the reflective support plate 110 may be formed to correspond to the bent shape of the reflector 80 to stably support the reflector 80.

The reflective support plate 110 may include a first reflective support plate 111 and a second reflective support plate 112 connected from one side of the first reflective support plate 111 in a direction intersecting the first reflective support plate 111. The reflective support plate 110 may include a third reflective support plate 113 connected from the other side of the first reflective support plate 111 to form an obtuse angle with the first reflective support plate 111.

The first reflective support plate 111 to the third reflective support plate 113 may extend along the extension direction of the sterilizing lamp 70. In other words, the reflective support plate 110 may extend corresponding to the extension direction of the reflector plate 80.

The second reflective support plate 112 and the second reflector 82 may be coupled to each other, and the third reflective support plate 113 and the third reflector 83 may be coupled to each other. According to such a structure, despite of the bent shape of the reflector 80, the reflector 80 may be supported more stably by the reflective support plate 110.

The lower rear frame 101 may include an inverter groove 120a. The inverter groove 120a may accommodate the inverter 120 inserted through an opening formed on a side of the inverter cover 91. That is, before the inverter 120 is inserted into the inverter groove 120a, the inverter groove 120a may be arranged on one side of the inverter 120, and the inverter cover 91 may be arranged on the other side of the inverter 120.

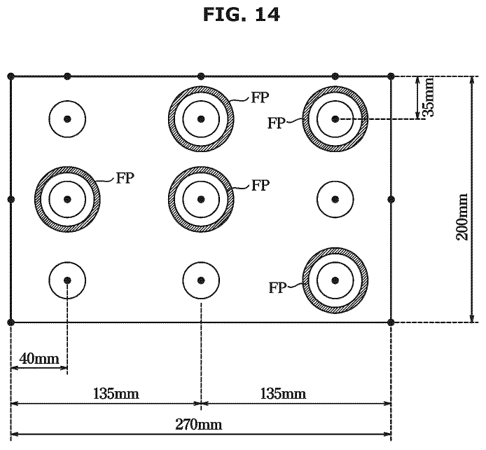

FIG. 14 is a drawing showing a fiber material of a filter 50 inoculated with a bacterial solution to show a sterilization experiment with a structure according to an embodiment of the disclosure.

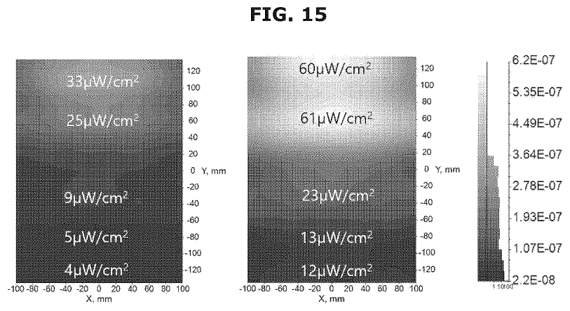

FIG. 15 is a drawing comparing the amount of light when the fiber material of the filter 50 shown in FIG. 14 is irradiated with the sterilizing lamp 70, see FIG. 11, and the amount of light when irradiated with the sterilizing lamp 70 and the reflector 80 according to an embodiment of the disclosure.

Referring to FIG. 14, the effect of the UVC irradiation on the sterilizing power of a rectangular fiber material, which is the same material as the filter 50, see FIG. 4 according to an example will be described.

The fiber material of the same material as the filter 50 may be provided in a rectangular shape with a width of 270 mm and a height of 200 mm. The fiber material may include nine points, with three points arranged horizontally and three points arranged vertically, and the three horizontal points may be arranged to be spaced apart from each other by a length of 90 mm. The three vertical points may be arranged to be spaced apart from each other by a length of 70 mm.

The experiment was conducted by inoculating a Staphylococcus aureus bacterial solution at five random points among the nine points to be sufficiently permeated, and then emitting UVC for a certain period of time. After that, inoculation parts FP were cut to surround the five points in which the bacterial solution had permeated in the fiber material of the filter 50, placed in a test buffer solution, and shaken to measure the concentration of the bacteria, thereby calculating the sterilizing power based on a decrease in the concentration of the bacterial solution compared to the concentration of the initial bacterial solution.

Accordingly, after UVC irradiation for 2.5 hours and 5 hours, the sterilizing powers for the five random points reached 99.86% and 99.97%, respectively, obtaining the following experimental results.

TABLE 1

| | | |
|---|---|---|
| UVC irradiation time (hr) | 2.5 | 5 |
| Sterilization power (%) | 99.86 | 99.97 |

Table 1. Experimental results showing sterilizing power according to the UVC irradiation time That is, when UVC is irradiated to a fiber material, which is the same material as the filter 50, for 5 hours or longer using the structure according to an example, a sterilizing power of 99.97% is achieved.

In addition, referring to FIG. 15, the effect of irradiating UVC on the fiber material shown in FIG. 14 is described by comparing UVC irradiation using only the sterilizing lamp 70 with UVC irradiation using the sterilizing lamp 70 and the reflector plate 80.

When the fiber material is irradiated with UVC using only the sterilizing lamp 70, the minimum amount of light is 4 μW/cm², and the maximum amount of light is 33 μW/cm² along the center line. When the fiber material is irradiated with UVC using the sterilizing lamp 70 and the reflector 80, the minimum amount of light amount is 12 μW/cm², and the maximum amount of light is 60 μW/cm² along the center line.

That is, when fiber material is irradiated with UVC using only the sterilizing lamp 70, the ratio of the maximum light amount to the minimum light amount is approximately 8, but when fiber material is irradiated with UVC using the sterilizing lamp 70 and the reflector plate 80, the ratio of the maximum light amount to the minimum light amount may be approximately 5.

Therefore, since the ratio of the maximum light amount to the minimum light amount when using the sterilizing lamp 70 and the reflector 80 is smaller, the ventilation apparatus 1 including the sterilizing lamp 70 and the reflector plate 80 may have more uniform irradiation power.

In addition, since the minimum light increases from 4 μW/cm² when only using the sterilizing lamp 70 to 12 μW/cm² when using the sterilizing lamp 70 and the reflector 80, it can be seen that the light reaching the fiber material farthest from the sterilizing lamp 70 increases.

The above described structure may be included not only in the ventilation apparatus 1, but also in a corresponding structure for an air conditioner or air cleaner.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A ventilation apparatus comprising:
a housing;
an inlet formed on one side of the housing such that outside air is introduced into the housing;
an outlet formed on another side of the housing such that the air introduced into the housing through the inlet is discharged to indoors;
a total heat exchanger arranged between the inlet and the outlet, and including an intake inlet end into which air introduced through the inlet and flowing toward the outlet is introduced;
a filter arranged inside the housing and arranged between the inlet and the total heat exchanger; and
a sterilizing lamp configured to sterilize the filter,
wherein the filter includes a first side extending in a vertical direction and a second side extending in a direction perpendicular to the first side, and
wherein the sterilizing lamp is arranged between one end of the filter in a direction in which the second side extends and one end of the intake inlet end in the direction in which the second side extends.

2. The ventilation apparatus of claim 1, wherein the sterilizing lamp extends in a direction in which the first side of the filter extends.

3. The ventilation apparatus of claim 2,
wherein the filter includes a plurality of crests extending toward the total heat exchanger and a plurality of troughs arranged between the plurality of crests, and
wherein the plurality of crests and the plurality of troughs extend in the direction in which the second side extends.

4. The ventilation apparatus of claim 1, further comprising:
a reflector configured to reflect light emitted from the sterilizing lamp to the filter, being bent to surround the sterilizing lamp and extending along the direction in which the first side extends.

5. The ventilation apparatus of claim 4, wherein the reflector includes a first reflector, a second reflector bent from one side of the first reflector, and a third reflector bent from another side of the first reflector.

6. The ventilation apparatus of claim 5, wherein the first reflector and the third reflector are arranged to form an obtuse angle with each other.

7. The ventilation apparatus of claim 6,
wherein an angle formed by the first reflector and the second reflector is between 70 degrees and 110 degrees, and
wherein an angle formed by the first reflector and the third reflector is between 110 degrees and 165 degrees.

8. The ventilation apparatus of claim 6,
wherein the sterilizing lamp includes a cylindrical shape, and
wherein, when a vertical distance between a center of the sterilizing lamp and the filter is denoted as h, and a length of the first reflector in a direction perpendicular to an extension direction of the first side is denoted as n and a length of the second reflector is denoted as p, n/h and p/h are each provided between 0.05 and 0.2.

9. The ventilation apparatus of claim 6,
wherein the sterilizing lamp includes a cylindrical shape, and
wherein, when a vertical distance between a center of the sterilizing lamp and the filter is denoted as h, and a length of the third reflector in a direction perpendicular to an extension direction of the first side is denoted as q, q/h is provided between 0.08 and 0.4.

10. The ventilation apparatus of claim 2,
wherein the sterilizing lamp includes a cylindrical shape, and
wherein, when a vertical distance between a center of the sterilizing lamp and the filter is denoted as h, and a length of the second side of the filter is denoted L, h/L is provided between 0.15 and 0.6.

11. The ventilation apparatus of claim 2,
wherein the sterilizing lamp includes a cylindrical shape, and
wherein, when a vertical distance between a center of the sterilizing lamp and the filter is denoted as h, a vertical distance between a vertical line from the center of the sterilizing lamp to an extension line of the second side of the filter and an end of the filter adjacent to the vertical line is denoted as m, h/m is provided between −0.2 and 0.5.

12. The ventilation apparatus of claim 4, further comprising:
a front frame including an opening that is open toward the filter, and a rear frame configured to support the front frame and the reflector and couplable to the housing.

13. The ventilation apparatus of claim 12, wherein the rear frame includes a reflective support plate formed to correspond to a bent shape of the reflector to support the reflector.

14. The ventilation apparatus of claim 12, further comprising:
a grid frame provided in the opening.

15. The ventilation apparatus of claim 12, wherein the housing includes an inner wall arranged at one side of the housing adjacent to the inlet and including a fixing groove into which one side of the rear frame adjacent to the filter is inserted.
